(19) United States
(12) Patent Application Publication
(10) Pub. No.: US 2019/0374359 A1
(43) Pub. Date: Dec. 12, 2019

(54) TIP CLAMP ASSEMBLY AND CONTROLLED EXPANSION STENT GRAFT DELIVERY SYSTEM USING SAME

(71) Applicant: Cook Medical Technologies LLC, Bloomington, IN (US)

(72) Inventors: Ryan Bradway, Tacoma, WA (US); Charles Baxter, West Lafayette, IN (US)

(21) Appl. No.: 16/417,830

(22) Filed: May 21, 2019

Related U.S. Application Data
(60) Provisional application No. 62/683,283, filed on Jun. 11, 2018.

(51) Int. Cl. A61F 2/966; A61F 2/07

(52) U.S. Cl. CPC A61F 2/966; A61F 2002/065; A61F 2/07

(57) ABSTRACT

A controlled expansion stent graft delivery system has an adjustment configuration in which a retractable sheath is at a retracted position out of contact with a stent graft, but expansion of the stent graft is controlled by a control tether, which has a middle segment wrapped around a fabric tube of the stent graft. The stent graft changes diameter responsive to a tension level in the control tether. At least one of an orientation and a position of the stent graft may be adjusted during controlled expansion via the control tether. After proper placement of the stent graft, a tip clamp assembly can be actuated to unclamp and release the control tether.

### TECHNICAL FIELD

The present disclosure relates generally to a catheter tip clamp assembly, and more particularly to controlled expansion of stent grafts that may have orientation and/or positioning sensitivities.

### BACKGROUND

Some variability is involved with placing stent grafts when un-sheathing and allowing full expansion utilizing known self-expanding stent designs. Some stent grafts, such as those located in the aorta, require precise placement and are often delivered by unsheathing the device to a secondary constrained diameter. From this point, the clinician may interpolate the stent graft's likely landing zone, and release the stent graft to self-expand to its final diameter opposed to a vessel wall. This process can introduce variability in a final landing zone due to the jump that occurs between the intermediate and final diameters in which the device is not constrained to the delivery system. During this brief period of time, blood flow and other factors can impact the trajectory of the stent graft changing its final landing zone. Problems can occur when the final landing zone is different from the intended landing zone for the stent graft, either in position or orientation, or both.

The present disclosure is directed toward one or more of the problems set forth above.

### SUMMARY

A catheter tip clamp assembly includes a clamp base with a first clamping surface and a first spring attachment, and defining a first clamp release passage. An indexing ring is configured to rotate with respect to the clamp base about a longitudinal axis, and defines a second clamp release passage and includes a second spring attachment. A spring is mounted about the clamp base and is attached at one end at the first spring attachment and at an opposite end at the second spring attachment. The spring includes a second clamping surface positioned opposite the first clamping surface. The indexing ring is rotatable about the longitudinal axis between a first orientation at which the clamp release passage is in registry with the second clamp release passage, and a second orientation at which the first clamp release passage is out of registry with the second clamp release passage. The first clamping surface is closer to the second clamping surface in the first orientation relative to the second orientation.

In another aspect, a controlled expansion graft delivery system includes a delivery catheter with a tip clamp assembly that includes a clamp base, a spring and an indexing ring, and defines a longitudinal axis. This system also includes a retractable sheath and a stent graft that includes a fabric tube attached to, and supported by, a self expanding stent. A handle is attached to the delivery catheter at an end opposite to the tip clamp assembly. A control tether extends between the tip clamp assembly and the handle. A clamp release line extends between the tip clamp assembly and the handle. The delivery system has a delivery configuration in which the stent graft is mounted on the delivery catheter in a compressed state and covered by the retractable sheath. The delivery system has an adjustment configuration in which the retractable sheath is at the retracted position out of contact with the stent graft, the control tether is held by the tip clamp assembly, wrapped around the stent graft, and the clamp release line holds the tip clamp assembly in a clamping configuration. A delivery system has a release configuration in which the control tether is released from the tip clamp assembly and wrapped around the stent graft, which is in an expanded state, and the clamp release line is moved to release the tip clamp assembly to an unclamped configuration. The delivery system has a detached configuration in which the stent graft is in the expanded state and the control tether is out of contact with the stent graft. The spring unwinds when the tip clamp assembly changes from the clamping configuration to the unclamped configuration.

In still another aspect, a method of operating a controlled expansion stent graft delivery system includes positioning the system at a treatment site in a delivery configuration in which the stent graft is mounted on the delivery catheter in a compressed state and covered by the retractable sheath. The delivery system is changed to an adjustment configuration in which the retractable sheath is at a retracted position out of contact with the stent graft, and the control tether is held by the tip clamp assembly, wrapped around the stent graft, and the clamp release line holds the tip clamp assembly in a clamping configuration. The delivery system is changed to a release configuration in which the control tether is released from the tip clamp assembly, wrapped around the stent graft, and the stent graft is in an expanded state, and the clamp release line has moved to release the tip clamp assembly to an unclamped configuration. The delivery system is changed to a detached configuration in which the stent graft is in the expanded state and the control tether is out of contact with the stent graft. The spring unwinds when the tip clamp assembly changes from the clamping configuration to the unclamped configuration.

### BRIEF DESCRIPTION OF THE DRAWINGS

FIG. 1 is an enlarged side view of a stent graft and control tether assembly mounted on the delivery system, which is partially shown;

FIG. 2 is a sectioned view through the assembly of FIG. 1 as viewed along section lines 2-2;

FIG. 3 is a schematic view of the control tether being tensioned between reduced and enlarged diameter configurations;

FIG. 4 is a close up perspective view through one of the loops of FIG. 1;

FIG. 5 is a partial exploded view of the tip of a delivery catheter for the delivery system of FIG. 1;

FIG. 6 is a partial perspective view of the tip clamp assembly for delivery system of FIG. 1 in a clamping configuration;

FIG. 7 is a partial sectioned view through the tip clamp assembly of FIG. 5 in a clamping configuration;

FIG. 8 is a partial sectioned view of through the tip clamp assembly in an unclamped configuration;

FIG. 9 is a schematic view of a handle for the delivery system of FIG. 1;

FIG. 10 is a schematic section view through the axial movement actuator and a portion of the handle along section lines 10-10 of FIG. 9;

FIG. 11 is a schematic section view similar to FIG. 10 except showing the axial movement actuator unlocked for movement;

FIG. 12 is a schematic view of a controlled expansion stent graft delivery system being maneuvered through an aorta in a delivery configuration;

FIG. 13 shows the delivery system moved from the delivery configuration of FIG. 10 to an adjustment configuration;

FIG. 14 is a schematic view of the delivery system in the adjustment configuration with the stent graft expanded;

FIG. 15 is a schematic view of the delivery system an adjustment configuration with the stent graft partially expanded;

FIG. 16 is a schematic view of the delivery system of FIG. 10 in a detached configuration; and

FIG. 17 is a schematic view of two more version of delivery system according to the present disclosure.

### DETAILED DESCRIPTION

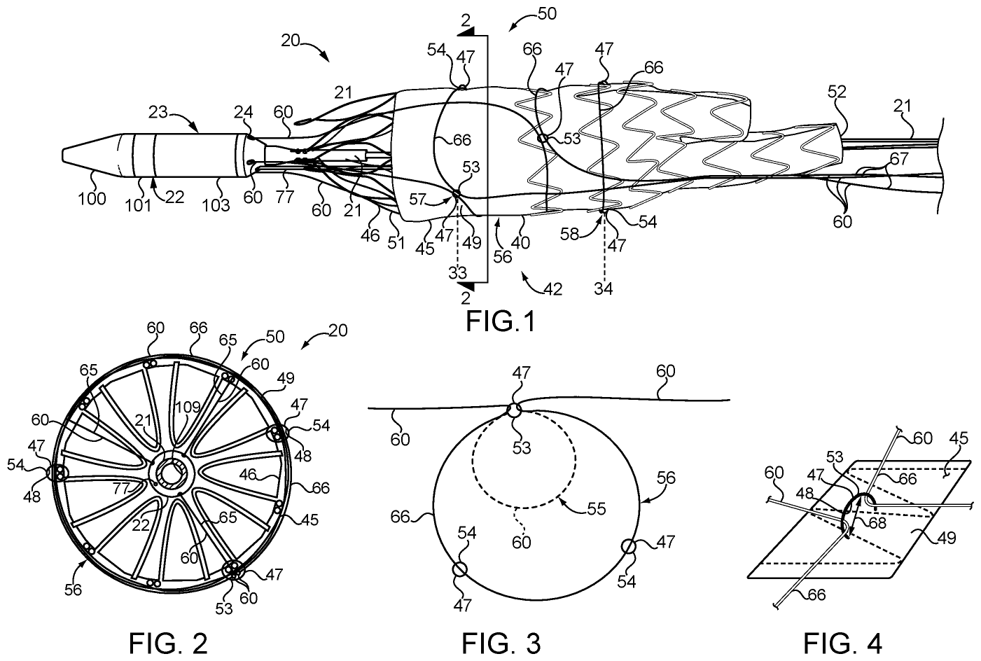

Referring initially to FIGS. 1-11, a controlled expansion stent graft delivery system 20 is utilized to deliver a stent graft 40 to a treatment location in a patient. In the illustrated embodiment, stent graft 40 includes a fabric tube 45 that is attached to, and supported by, a self expanding stent 46, and is shaped for treatment of an abdominal aortic aneurism in a manner known in the art. Stent graft 40 is a portion of an assembly 50 that includes at least one control tether 60. Apart from a stent graft and control tether assembly 50, the delivery system 20 includes a delivery catheter 21 with a tip 22 that includes a tip clamp assembly 23. A retractable sheath 30 is slidably movable with regard to the delivery catheter 21 in a manner well known in the art. In addition to the sutures that may or may not be included to attach the fabric tube 45 to the self expanding stent 46, the stent graft 40 includes a plurality of loops 47 that are attached to at least one of the fabric tube 45 and the self expanding stent 46. Loops 47 may be formed with suture material. Each of the loops 47 has an opening 48 exposed on a radially outward side 49 of fabric tube 45. Each of three control tether(s) 60 in the illustrated embodiment includes a first segment 65, a middle segment 66 and a second segment 67. Each middle segment 66 is wrapped around the fabric tube 45 and is received through the respective openings 48 of the loops 47 at each of three different longitudinal positions along stent graft 40. The first segment 65 of each of the control tethers 60 extends beyond one end 51 of the fabric tube 45. Each of the second segments 67, which are longer than the respective first segments 65, extends beyond an opposite end 52 of the fabric tube 45.

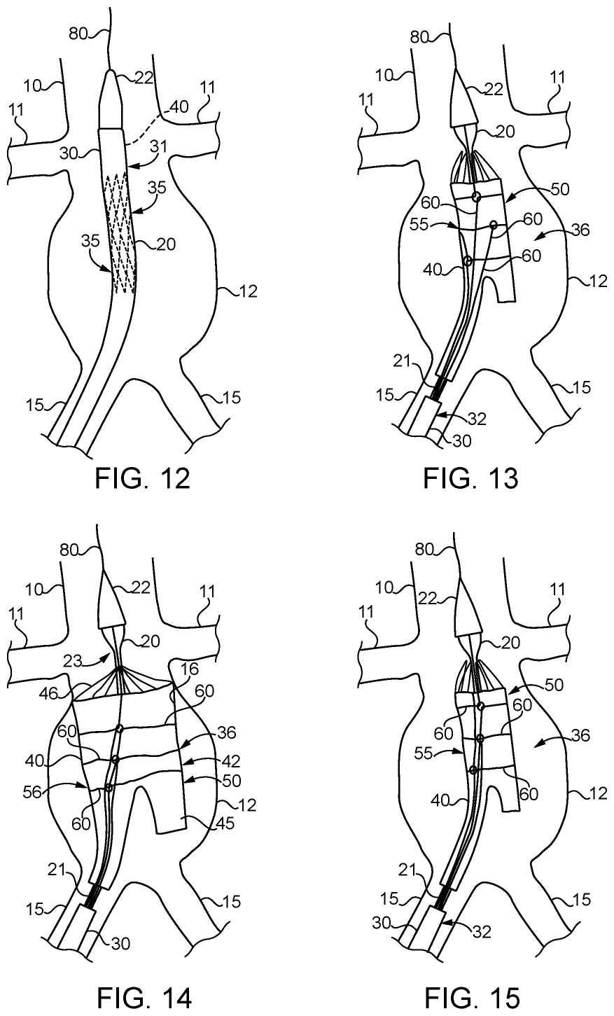

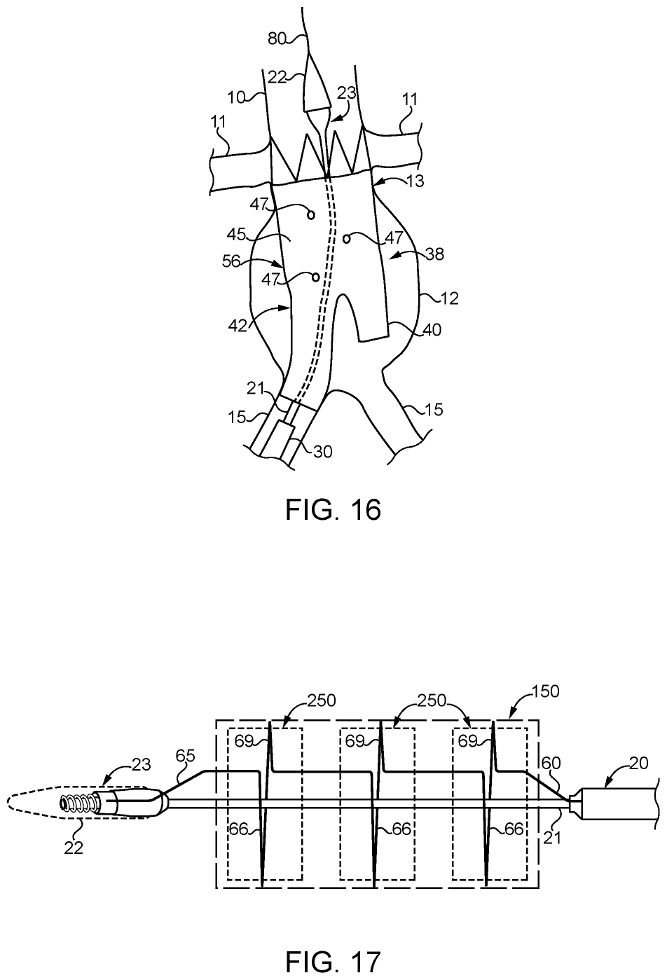

Referring in addition to FIGS. 12-16, delivery system 20 has a delivery configuration 35 in which the stent graft 40 is mounted on the delivery catheter 21 in a compressed state 41 and covered by the retractable sheath 30, which is at a covering position 31 (FIG. 12). The delivery system 20 is movable from the delivery configuration 35 shown in FIG. 12 to an adjustment configuration 36 (FIGS. 13-15) in which tension in the control tether(s) 60 keeps the stent graft 40 from fully expanding to allow the user to adjust a position and/or an orientation of the stent graft 40 at the delivery site. The delivery system 20 may be moved from the adjustment configuration 36 to a release configuration 37 (FIG. 8) with the stent graft in an expanded state 42 after the positioning and orientation of the stent graft 40 have been adjusted by the user to a desired landing zone in the treatment vessel. The release configuration is characterized by the control tether 60 being released from tip clamp assembly 23 but still wrapped around stent graft 40. Thereafter, the delivery system 20 may be movable from the release configuration 37 to a detached configuration 38 (FIG. 16) in which the stent graft 40 is in the expanded state 42 and the control tether(s) 60 is pulled toward handle 106 and out of contact with the stent graft 40. Thus, the use of the control tether(s) 60 allows the user to controllably expand, shrink and re-expand the stent graft 40 at the delivery site to better position the stent graft 40 at a desired position and orientation within a patient.

In the example embodiment shown in FIGS. 1 and 2, stent graft 40 includes a first set 57 of three loops 47 located at a first longitudinal location 33, and a second set 58 of at least three loops 47 at a second longitudinal location 34 that is spaced along the longitudinal axis from the first set of loops 57. In addition, a third set of at least three loops 47 is located at a longitudinal position between the first longitudinal location 33 and the second longitudinal location 34. Although each longitudinal location has three loops 47, one, two or four or more loops at each location would also fall within the scope of this disclosure. These spaced apart longitudinal positions are chosen such that when the tension is applied to the control tethers 60, the respective middle segments 66 can be used to change the self expanding stent 46 between a reduced diameter configuration 55 (FIG. 3) when the control tether(s) 60 is in tension, and an enlarged diameter configuration 56 (FIGS. 1-3) when the control tether(s) 60 has little or no tension. As best shown in FIG. 4, the opening 48 defined by each of the loops 47 has a diameter 68 at least several times larger than the diameter of the respective control tether 60 so that the control tether 60 is freely slidable through the respective opening 48. In the illustrated embodiment, the loops 47 have been shown added to a conventional AAA bifurcated main body graft of the type manufactured by Cook Inc., but the present disclosure is also applicable to stent grafts for virtually any treatment application, especially those requiring precise placement and orientation. Each of the respective control tethers 60 is received through one of the loops 53 in the respective set of loops 57 or 58 exactly twice and received through the other loops 54 of that respective set of loops exactly once. This feature is shown for example in FIG. 2, and partially shown in FIGS. 1, 3 and 4.

When delivery system 20 is in either the delivery configuration 35 (FIG. 12) and an adjustment configuration 36 (FIGS. 13-15) to be described infra, the first segment 65 of each of the respective control tethers 60 is held by the tip clamp assembly 23, which forms a portion of tip 22. Thus, by jailing first segments 65, a user can apply tension to the remote end of second segment 67 by moving an axial movement actuator 108 mounted to move on the handle 106, to control shrinkage and expansion of the stent graft 40 between the reduced diameter configuration 55 and the enlarged diameter configuration 56. The stent graft 40 changes its diameter responsive to a tension level in the control tether(s) 60 when the delivery system 20 is in its adjustment configuration 36. The user may actually reduce the diameter of stent graft 40 responsive to an increase in the tension level of the control tether(s) 60 when the delivery system 20 is in the adjustment configuration 36. The tension level in the control tether 60 acts in opposition to the spring tendency of the self expanding stent 46 to expand.

Referring now specifically to FIGS. 9-11, the delivery system 20 includes a handle 106 attached to delivery catheter 21 at an end 107 opposite to the tip 22. The remote end of control tether(s) 60 is attached to an axial movement actuator 108 mounted to handle 106. The axial movement actuator 108 is constrained to move within a guiding slot 87 that include guide surfaces defined by portions of handle 106. Guide slot 87 may be aligned with the longitudinal axis of catheter 21. In the illustrated embodiment, the axial movement actuator 108 can be stopped in perpendicularly oriented notches 84, 85 and 86, which correspond to different positions. For instance, the delivery system could initially be set up with the axial position actuator 108 in the notch 84 corresponding to a first position 81 such that the control tether(s) 60 would not interfere with full radial expansion of the stent graft 40 at time of implantation. However, if the stent graft 40 lands at an undesirable location or orientation, the user may then move the axial movement actuator 108 toward notches 85 or 86 (adjustment configuration 36) in order to apply tension to control tether(s) 60 to reduce the diameter of the stent graft 40 to make it possible to readjust at least one of its position and orientation at the implantation site. Thereafter the user can again move the axial position actuator 108 toward the first notch 84 in order to allow for full expansion of the stent graft 40 at the new landing location. The axial position actuator 108 may include a position lock 90 so that the axial position actuator can be locked into one of the notches 84-86, or maybe even at a desired location in guide slot 87. This may be accomplished by a threaded end 91 of the axial position actuator 108 being threadably received in a block 92, with a wall of the handle 106 sandwiched therebetween. By tightening threads 91 into block 92, the wall of the handle 106 can be clamped therebetween to hold the control tether 60 in a desired placement and tension state, corresponding to the position lock 90 being in a locked state. By untightening threads 91 from block 92 the position lock is placed in a unlocked state, and the axial position actuator 108 may be moved to any desired location constrained by guide slot 87, and then may be stopped at that new location by again locking position lock 90. The remote end of control tether 60 may be attached to move with block 92 as shown in FIGS. 10 and 11.

Position lock 90 could take on other forms and structures known in the art for allowing and locking against relative movement in medical devices. Although FIG. 9 shows the axial position actuator 108 initially positioned at a first position in notch 84, the system could also be set up initially to have the axial position actuator in an optional initial position 83 corresponding to notch 86. In such a case, the control tether 60 would initially be in tension to prevent the stent graft 40 from fully expanding after the retractable sheath 30 is moved to its retracted position 32. In such an instance, the operator could then manipulate the delivery system and the control tether via the axial movement actuator 108 to controllably land the stent graft 40 at a desired location and orientation at a delivery site. Thus, the delivery system 20 of the present disclosure can be set up initially to allow the operator to only use the control tethers if the stent graft 40 lands at an undesirable location or orientation, or may allow the user to control the expansion of the stent graft 40 prior to any landing.

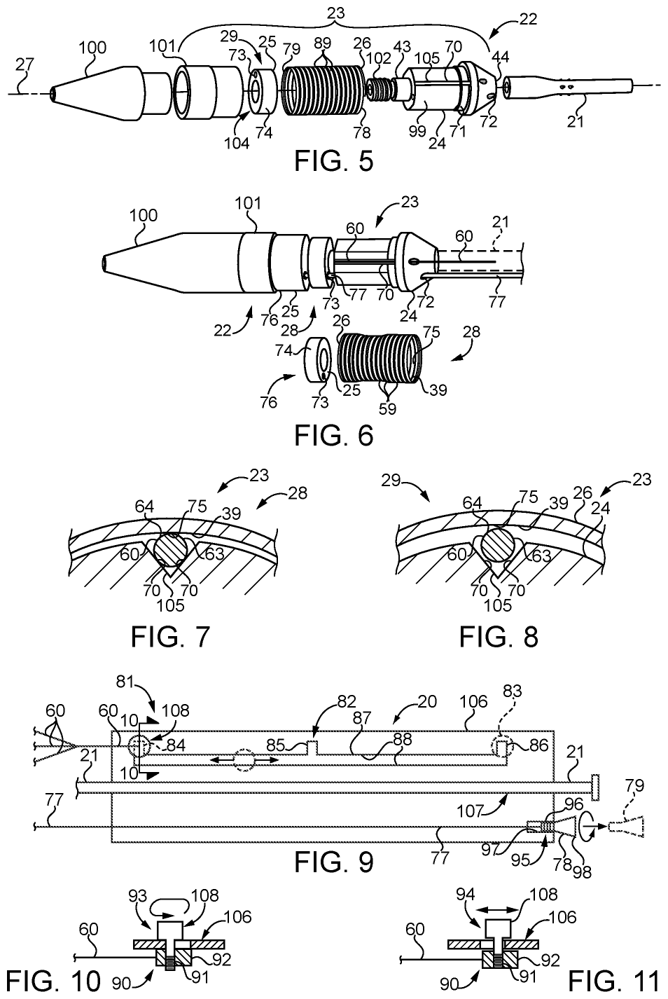

A remote end of the clamp release line 77 may be attached to a clamp release lock 95 such that the clamp release line 77 extends between the tip clamp assembly 23 (FIG. 5-8) and the handle 106 (FIG. 9). In the illustrated embodiment, clamp release lock 95 operates by including a threaded end 96 received in a threaded bore 97 defined by handle 106. Thus, when the clamp release lock 95 is threadably received in threaded bore 97 as shown in FIG. 9, the clamp release actuator 98 is inoperable. FIG. 9 also shows the dotted lines after the clamp release actuator 95 has been unthreaded from threaded bore 97 and moved backwards to move the tip clamp 23 from its clamping configuration 28 (FIG. 7) to the unclamped configuration 29 (FIG. 8). Numerals 28 and 29 appear in FIGS. 7-9 to show the relationship between clamp release actuator 98 and the positioning of tip clamp assembly 23. Movement of the tip clamp assembly may be enabled by unthreading clamp release actuator 98 from threaded bore 97 in handle 106 to allow the clamp release actuator to be moved away from handle 106 to move the tether clamp to its unclamped configuration 29. When a tip clamp assembly 23 is in the unclamped configuration 29, the axial movement actuator 108 may be moved in the direction of notch 86 to move the remote end of control tether 60 away from tip clamp assembly 23.

Referring specifically to FIGS. 5-8, the tip clamp assembly 23 includes a clamp base 24 that includes a first clamping surface 70 and a first spring attachment 71. Clamp base 24 also defines a first clamp release passage 72. An indexing ring 25 is configured to rotate with respect to the clamp base about a longitudinal axis 27. This may be accomplished by supporting indexing ring 25 for rotation by a bearing surface 43 of clamp base 24. The indexing ring includes a second clamp release passage 73 and a second spring attachment 74. A spring 26 is mounted about clamp base 24 and is attached at one end 78 at the first spring attachment 71 and at an opposite end 79 at a second spring attachment 74. The spring includes a second clamping surface 75 that is positioned opposite the first clamping surface 70. Second clamping surface 75 may include a plurality of discontiguous surfaces 59 located on respective turns 89 of the spring 26. In particular, the second clamping surface 75 is a portion of an inner surface 39 of spring 26. This is to be contrasted with the first clamping surface 70 which is a portion of an outer surface 99 of clamp base 24. The second clamping surface 75 may include at least three turns 89 of spring 26. The indexing ring 25 is rotatable about longitudinal axis 27 between a first orientation 76 at which the first clamp release passage 72 of clamp base 24 is in registry with the second clamp release passage 73 as shown in FIG. 6, and a second orientation 77 at which the first clamp release passage is out of registry with the second clamp release passage as shown in FIG. 5. As shown in FIGS. 7 and 8, the first clamping surface 70 is closer to the second clamping surface 75 and the first orientation 76 relative to the second orientation 77. Preferably, the first orientation 76 is less than one full rotation of spring 26 about longitudinal axis 27 from the second orientation 77. The second orientation 77 corresponds to a rest shape for spring 26. When indexing ring 25 is rotated to the first orientation 76, stress is stored in spring 26 as its turns move closer to clamp base 24 to clamp control tether 60. The clamp release line 77 extends through the first clamp release passage of clamp base 24 and the second clamp release passage 73 of indexing ring 25 to hold the stored stress in the spring 26 in the clamping configuration 28 of the first orientation 76. The clamp release line 77 is pulled out of the second clamp release passage 73 of the indexing ring 25 to allow the indexing ring to rotate responsive to the stored stress to the second orientation 77 in the unclamped configuration 29. Thus, the control tethers 60 are positioned between the first clamping surface 70 and the second clamping surface 75 to manipulate the stent graft 40 in the clamping configuration 28, but may be pulled clear of the stent graft 40 as shown in FIG. 16 in the unclamped configuration 29 after the stent graft has been properly positioned in the patient. Thus, one could expect the spring 26 to unwind responsive to withdrawal of the clamp release line 77 from indexing ring 25 to allow the tip clamp assembly 23 to change from its clamping configuration 28 to the unclamped configuration 29.

Although not necessary, each control tether 60 may be a monofilament wire 63, with the tip clamp assembly 23 constructed to contact the monofilament wire 63 at three spaced apart locations around a circumference 64 of the monofilament wire 63 as best shown in FIG. 7. In order to have the three point clamping configuration as shown in FIG. 7, clamp base 24 may define a v-shaped groove 105 that includes two of three clamping surfaces 70 that contact the monofilament wire 63 at two of three spaced apart locations on the outer surface 99 of the clamp base 24. The spring 26 includes a clamping surface 75 on an inner surface 39 of the spring 26 that contacts the monofilament wire 63 as shown at FIG. 7. Those skilled in the art will appreciate that other clamping configurations could be utilized. For instance, groove 105 could be omitted so that clamping surface 70 were on a cylindrical surface of clamp base 24. The control tether(s) could be multifilament instead of the monofilament wire 63 of the illustrated embodiment.

Referring now in addition to FIG. 17, a couple of different variations of a stent graft and control tether assembly 150 and 250 are shown. With regard to assembly 150, a single stent graft is schematically shown and has a single control tether 60 wrapped around the stent graft so that middle segment 66 wraps around the fabric tube a first time and a second time 69 and the third time, thus illustrating that each control tether may wrap around an individual stent graft more than one time at different longitudinal locations along the stent graft. Also shown, is an assembly 250 in which three separate stent grafts share a common single control tether 60 such that each of the three stent grafts has a middle segment 66 of the control tether 60 wrapped one time around each of the three stent grafts. Thus, those skilled in the art will appreciate that the present disclosure is applicable to delivery assemblies for delivering more than one stent graft that share a common control tether and to a single stent graft that uses only one control tether wrapped around the stent graft more than one time. This is to be contrasted with the illustrated embodiment in which three control tethers 60 are used with each of the control tethers 60 being wrapped around the stent graft 40 one time. Those skilled in the art will appreciate that other permutations would also fall within the scope of this disclosure.

### INDUSTRIAL APPLICABILITY

The present disclosure finds general applicability to any medical device that could benefit from a tip clamp assembly. The present disclosure finds particular applicability to stent grafts with sensitivities to placement location and/or orientation, such as for accommodating branch vessels. The present disclosure finds specific applicability for delivering stent grafts to high flow areas, such as those associated with the aorta, which also include many branching arteries that must be accommodated by proper placement and orientation of a stent graft.

Referring now in addition to FIGS. 12-16, an example method of operating a controlled expansion stent graft delivery system 20 is shown for treating an aneurysm 12 in the aorta 10 that is located immediately below branching vessels, which may include the renal artery 11. The delivery system 20 may approach the implantation site utilizing a previously placed wire guide 80 utilizing an access in the patient's leg (not shown) and travel up through the iliac artery 15. Wire guide 80 may be received through a lumen 109 of delivery catheter 21. In particular, the delivery system 20 is positioned at a treatment site in a delivery configuration 35 in which the stent graft 40 is mounted on delivery catheter 21 in a compressed state 41 and covered by retractable sheath 30, which is at a covering position 31. Next, as shown in FIG. 13, the delivery system is moved to the adjustment configuration 36 with the retractable sheath 30 moved from the covering position 31 shown in FIG. 10 to the retracted position 32 to uncover the stent graft 40. Because the stent graft 40 did not fully expand upon retraction of retractable sheath 30, this reveals that the control tether(s) 60 were configured with some initial tension by initially positioning axial position actuator 108 in notch 86 or in notch 85 depending upon the total movement length available for control tether 60 in a given diametrical application. When in the adjustment configuration 36, the control tethers 60 have sufficient tension to prevent the stent graft 40 from fully expanding. When in this configuration, the retractable sheath 30 is out of contact with the stent graft 40. However, the control tethers 60 are held by the tip clamp assembly 23, are wrapped around the stent graft 40, and received through the openings 48 in the individual loops 47 of the stent graft 40. The delivery system 20 may be changed from the delivery configuration of FIG. 12 to the adjustment configuration 36, thus allowing the user to make some orientation change via rotating delivery system 20 about its longitudinal axis 27 and positioning adjustments by advancing or retracting the delivery system 20 with regard to the aneurysm 12. When the user believes that the stent graft 40 is properly positioned, tension on the control tethers 60 may be relaxed by unlocking position lock 90 and moving axial position actuator 108 toward notch 84, and allow the self expanding stent 46 to move the stent graft 40 into contact with the vessel wall at an initial landing zone 16. The user can then use known visualization strategies to assess both the orientation and placement of stent graft 40 while the delivery system 20 is still in an adjustment configuration 36. The difference between the adjustment configuration 36 shown in FIG. 13 and that shown in FIG. 14 is associated with the level of tension in the control tether(s) 60 via the positioning of axial movement actuator 108. If the physician deems that the initial landing zone 16 is not acceptable, the tension level in the control tethers 60 may be increased to shrink the diameter of stent graft 40 as shown in FIG. 15. When in the adjustment configuration 36, the user may adjust the orientation and position of the stent graft 40, and then relieve tension on the control tether(s) 60 so that the stent graft 40 is controllably expanded to the desired landing zone 13 as shown in FIG. 16. After confirming the proper placement of stent graft 40, the physician may release the top stent proximal fixation that are adjacent the renal artery 11 in a manner well known in the art. Next, after confirming a proper seal, the delivery system 20 may be moved from the adjustment configuration 36 to a release configuration 37 (FIG. 8) in which the control tether 60 is released from the tip clamp assembly 23, but remains wrapped around the stent graft 40 and received through the openings 48 of the loops 47, and the stent graft 40 is in its expanded state 42. Changing to the release configuration may include movement of the clamp release lock 95 to an unlocked state by unthreading clamp release actuator 98 from threaded bore 97. Next, the clamp release actuator is moved away from handle 106 as shown in FIG. 9 to move clamp release line 77 out of indexing ring 25 to allow spring 26 to unwind to the unclamped configuration 29. When this is done, the axial movement actuator 108 may be moved slightly to move the remote end of control tether 60 out of tip clamp assembly 23 so that the control tether 60 can be moved out of contact with tip clamp assembly 23 and stent graft 40.

Thereafter, the delivery system 20 may then be moved from the release configuration 37 to a detached configuration 38 as shown in FIG. 16 in which the stent graft 40 is at the expanded state 42 and the control tether 60 is slid out of contact with the stent graft 40. This is accomplished because the first segment 65 of the individual control tether(s) have been released from tip clamp assembly 23, and withdrawn back through loops 47 toward handle 26 and out of contact with the stent graft 40. Next, the user may withdraw the delivery system 20 leaving the stent graft 40 implanted in a desired position and orientation.

Those skilled in the art will appreciate that while the delivery system 20 is in the adjustment configuration 36 as shown in FIGS. 13-15, the expansion of stent graft 40 may be stopped by increasing tension in the control tether(s) 60. The user may then adjust at least one of the orientation and position of the stent graft 40 while expansion of the stent graft 40 has been stopped. Thereafter, the expansion of the stent graft may be controllably resumed by decreasing tension in control tether 60. The expansion of the stent graft 40 may be stopped by securing the axial movement actuator 108 at any desired position using position lock 90. If the positioning and/or orientation are not as desired, the stent graft 40 may be contracted toward the compressed state 41 by increasing tension in the control tether 60 while the delivery system 20 is in the adjustment configuration 36. As discussed earlier, after proper placement, the tip clamp assembly 23 may be moved from its clamping configuration 28 to its unclamped configuration 29 responsive to withdrawal of the clamped release line 77.

It should be understood that the above description is intended for illustrative purposes only, and is not intended to limit the scope of the present disclosure in any way. Thus, those skilled in the art will appreciate that other aspects of the disclosure can be obtained from a study of the drawings, the disclosure and the appended claims.

What is claimed is:

1. A catheter tip clamp assembly comprising:
a clamp base that includes a first clamping surface and a first spring attachment, and the clamp base defines a first clamp release passage;
an indexing ring configured to rotate with respect to the clamp base about a longitudinal axis, and defining a second clamp release passage, and the indexing ring includes a second spring attachment;
a spring mounted about the clamp base and being attached at one end at the first spring attachment and at an opposite end at the second spring attachment, and the spring including a second clamping surface positioned opposite the first clamping surface;
wherein the indexing ring is rotatable about the longitudinal axis between a first orientation at which the first clamp release passage is in registry with the second clamp release passage, and a second orientation at which the first clamp release passage is out of registry with the second clamp release passage; and
wherein the first clamping surface is closer to the second clamping surface in the first orientation relative to the second orientation.

2. The catheter tip clamp assembly of claim 1 wherein the second clamping surface includes a plurality of discontiguous surfaces located on respective turns of the spring.

3. The catheter tip clamp assembly of claim 2 wherein the plurality of discontiguous surfaces include at least three turns of the spring.

4. The catheter tip clamp assembly of claim 1 wherein the first clamping surface is a portion of an outer surface of the clamp base; and
the second clamping surface is a portion of an inner surface of the spring.

5. The catheter tip clamp assembly of claim 1 wherein the first orientation is less than one full rotation from the second orientation.

6. The catheter tip clamp assembly of claim 1 including a control tether positioned between the first clamp surface and the second clamp surface.

7. The catheter tip clamp assembly of claim 1 including a control release line;
wherein the spring has stored stress at the first orientation;
the clamp release line extending through the first clamp release passage and the second clamp release passage at the first orientation to hold the stored stress in the spring in a clamping configuration;
the clamp release line being out of the second clamp release passage, the indexing ring has rotated responsive to the stored stress in the spring to the second orientation in an unclamped configuration.

8. The catheter tip clamp assembly of claim 1 wherein the indexing ring is supported for rotation by a bearing surface of the clamp base; and
the clamp base includes a catheter attachment surface.

9. A controlled expansion stent graft delivery system comprising:
a delivery catheter with a tip clamp assembly that includes a clamp base, a spring and an indexing ring, and defining a longitudinal axis;
a retractable sheath;
a stent graft that includes a fabric tube attached to, and supported by, a self expanding stent;
a handle attached to the delivery catheter at an end opposite to the tip clamp assembly;
a control tether extending between the tip clamp assembly and the handle;
a clamp release line extending between the tip clamp assembly and the handle;
the delivery system having a delivery configuration in which the stent graft is mounted on the delivery catheter in a compressed state and covered by the retractable sheath;
the delivery system having an adjustment configuration in which the retractable sheath is at a retracted position out of contact with the stent graft, and the control tether is held by the tip clamp assembly, wrapped around the stent graft, and the clamp release line holding the tip clamp assembly in a clamping configuration;
the delivery system having a release configuration in which the control tether is released from the tip clamp assembly and wrapped around the stent graft, which is in an expanded state, and the clamp release line moved to release the tip clamp assembly to an unclamped configuration;
the delivery system having a detached configuration in which the stent graft is in the expanded state and the control tether is out of contact with the stent graft; and
the spring unwinds when the tip clamp assembly changes from the clamping configuration to the unclamped configuration.

10. The delivery system of claim 9 wherein the clamp base includes a first clamping surface and a first spring attachment, and the clamp base defines a first clamp release passage;
an indexing ring configured to rotate with respect to the clamp base about a longitudinal axis, and defining a second clamp release passage, and the indexing ring includes a second spring attachment;
the spring mounted about the clamp base and being attached at one end to the first spring attachment and at an opposite end to the second spring attachment, and the spring including a second clamping surface positioned opposite the first clamping surface.

11. The delivery system of claim 10 wherein changing from the clamping configuration to the unclamped configuration is responsive to movement of the clamp release line from a position inside the second clamp release passage to a position outside the second clamp release passage.

12. The delivery system of claim 11 wherein the second clamping surface includes a plurality of discontiguous surfaces located on an inner surface of at least three turns of the spring; and
the first clamping surface is a portion of an outer surface of the clamp base.

13. The delivery system of claim 12 the spring unwinds less than one full rotation when the tip clamp assembly changes from the clamping configuration to the unclamped configuration.

14. The delivery system of claim 13 wherein the control tether is clamped between the first clamp surface and the second clamp surface in the adjustment configuration; and
the indexing ring rotates responsive to the stored stress in the spring to the from the clamping configuration to the unclamped configuration.

15. A method of operating a controlled expansion stent graft delivery system including a delivery catheter with a tip clamp assembly that includes a clamp base, spring and a tether clamp, and defining a longitudinal axis; a retractable sheath; a stent graft that includes a fabric tube attached to, and supported by, a self expanding stent; a handle attached to the delivery catheter at an end opposite to the tip clamp assembly; a control tether extending between the tip clamp assembly and the handle; a clamp release line extending between the tip clamp assembly and the handle, and the method comprising the steps of:
positioning the delivery system at a treatment site in a delivery configuration in which the stent graft is mounted on the delivery catheter in a compressed state and covered by the retractable sheath;
changing the delivery system to an adjustment configuration in which the retractable sheath is at a retracted position out of contact with the stent graft, and the control tether is held by the tip clamp assembly, wrapped around the stent graft, and the clamp release line holding the tip clamp assembly in a clamping configuration;
changing the delivery system to a release configuration in which the control tether is released from the tip clamp assembly, wrapped around the stent graft, and the stent graft is in an expanded state, and the clamp release line has moved to release the tip clamp assembly to an unclamped configuration; and
changing the delivery system to a detached configuration in which the stent graft is in the expanded state and the control tether is out of contact with the stent graft; and
the spring unwinds when the tip clamp assembly changes from the clamping configuration to the unclamped configuration.

16. The method of claim 15 wherein changing from the clamping configuration to the unclamped configuration is responsive to movement of the clamp release line from a position inside a second clamp release passage of an indexing ring of the tip clamp assembly to a position outside the clamp release passage.

17. The method of claim 16 wherein a second clamping surface of the tip clamp assembly includes a plurality of discontiguous surfaces located on an inner surface of at least three turns of the spring; and
a first clamping surface is a portion of an outer surface of the clamp base.

18. The method of claim 17 wherein the spring unwinds less than one full rotation when the tip clamp assembly changes from the clamping configuration to the unclamped configuration.

19. The method of claim 18 wherein the control tether is clamped between the first clamp surface and the second clamp surface in the adjustment configuration; and
the indexing ring rotates responsive to the stored stress in the spring to the from the clamping configuration to the unclamped configuration.

20. The method of claim 19 wherein the control tether is one of a plurality of control tethers.
